(19) United States
(12) Patent Application Publication
(10) Pub. No.: US 2022/0260505 A1
(43) Pub. Date: Aug. 18, 2022

(54) METHOD AND SYSTEM OF INSPECTING PRE-MOLDED SEALANT PARTS

(71) Applicants: PRC-DESOTO Intemational, Inc., Sylmar, CA (US); KA Imaging Inc., Kitchener (CA)

(72) Inventors: Soccorso Rizzello, Toronto (CA); Karim Sallaudin Karim, Waterloo (CA); Christopher Charles Scott, Waterloo (CA)

(73) Assignees: PRC-DESOTO Intemational, Inc., Sylmar, CA (US); KA Imaging Inc., Kitchener, ON (CA)

(21) Appl. No.: 17/618,159

(22) PCT Filed: Jun. 10, 2020
(86) PCT No.: PCT/US2020/037041

§ 371 Date: Dec. 10, 2021

Related U.S. Application Data
(60) Provisional application No. 62/859,744, filed on Jun. 11, 2019.

(51) Int. Cl. G01N 23/041; G01N 33/44

(52) U.S. Cl. CPC G01N 23/041; G01N 2223/646; G01N 2223/04; G01N 33/445

(57) ABSTRACT

A method and system of inspecting pre-molded parts comprising sealant material are disclosed. The method includes directing a micro-focused X-ray beam through a pre-molded sealant part comprising a sealant material to produce a phase-shifted refracted X-ray beam, and detecting the refracted X-ray beam with at least one phase contrast imaging X-ray detector to produce an image of the sealant material. The system includes at least one X-ray source structured and arranged to direct a micro-focused X-ray beam through a pre-molded sealant part comprising a sealant material to produce a phase-shifted refracted X-ray beam, and at least one phase contrast imaging X-ray detector structured and arranged to detect the refracted X-ray beam to produce an image of the sealant material.

### CROSS-REFERENCE TO RELATED APPLICATION

This application claims the benefit of U.S. Provisional Patent Application Ser. No. 62/859,744, filed on Jun. 11, 2019, which is incorporated herein by reference.

### FIELD OF THE INVENTION

The present invention relates to a method and system of inspecting pre-molded parts comprising sealant material.

### BACKGROUND OF THE INVENTION

Parts that have been formed by a molding process and which comprise a sealant material for sealing components such as mechanical fasteners are typically manually inspected for air bubbles and other defects. However, the manual inspection process is time-consuming and inconsistent.

### SUMMARY OF THE INVENTION

The present invention provides a method of inspecting a pre-molded sealant part to identify defects in the pre-molded sealant part. The method comprises directing a micro-focused X-ray beam through a pre-molded sealant part comprising a sealant material to produce a phase-shifted refracted X-ray beam, and detecting the refracted X-ray beam with at least one phase contrast imaging X-ray detector to produce an image of the sealant material.

The present invention further provides a system for inspecting a pre-molded sealant part to identify defects in the pre-molded sealant part comprising at least one X-ray source structured and arranged to direct a micro-focused X-ray beam through a pre-molded sealant part comprising a sealant material to produce a phase-shifted refracted X-ray beam, and at least one phase contrast imaging X-ray detector structured and arranged to detect the refracted X-ray beam to produce an image of the sealant material.

### BRIEF DESCRIPTION OF THE DRAWINGS

FIG. 1 is a schematic side sectional view of an outer shell of a seal cap.

FIG. 2 is a schematic side sectional view of an outer shell of a seal cap schematically showing defects in the outer shell.

FIG. 3 is a schematic diagram of a top view of an imaging system in accordance with the present invention.

FIG. 4 is a schematic diagram of a top view of an imaging system in accordance with the present invention.

FIG. 5 is a schematic diagram of a top view of an imaging system in accordance with the present invention.

FIG. 6 is a schematic diagram of a side view of an imaging system in accordance with the present invention.

FIG. 7 is a schematic diagram of a top view of an imaging system in accordance with the present invention.

FIG. 8 is a flow chart illustrating a method of phase contrast imaging a pre-molded sealant part in accordance with the present invention.

FIG. 9 is a photograph of an outer shell of a seal cap.

FIGS. 10 and 11 are grayscale images of the outer shell of the seal cap of FIG. 9 scanned with a system of the present invention.

### DETAILED DESCRIPTION

The present invention provides an imaging method and system for inspecting and evaluating pre-molded parts made from sealant material such as seal caps, gaskets, O-rings, shims, washers, grommets, spacers, packing, cushions, mating material, flanges, plugs, and the like. The sealant material may typically be in a fully cured condition but may also be partially cured. As used herein, the term “pre-molded sealant part” refers to parts that have been formed from a sealant material into a predetermined shape and at least partially cured to retain that shape. Although the parts are referred to herein as being “pre-molded”, the parts can be made by any suitable method, such as molding, extrusion, additive manufacturing, 3D printing and the like. As used herein, the terms “sealant material” and “sealant” include any known type of sealants and adhesives that may be used for various applications, including the aerospace industry, the automotive industry, and other industries, and have the ability, when cured, to resist atmospheric conditions such as moisture and temperature and at least partially block the transmission of materials such as water, water vapor, fuel, solvents, and/or liquids and gases.

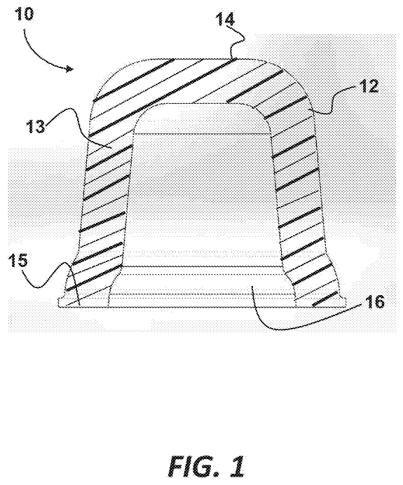

FIG. 1 illustrates a pre-molded seal cap 10 that may be inspected in accordance with the present invention. The seal cap 10 includes an outer shell 12 having a generally cylindrical or conical sidewall 13, a top 14, and a bottom rim 15. A recessed sealant reservoir 16 is provided near the bottom of the outer shell 12 radially inside the bottom rim 15. An uncured or partially uncured adhesive or sealant may be introduced into the seal cap assembly by filling the recessed sealant reservoir with an uncured sealant. U.S. Pat. No. 7,438,974 issued Oct. 21, 2008, U.S. Pat. No. 9,447,808 issued Sep. 20, 2016, and U.S. Pat. No. 9,533,798 issued Jan. 3, 2017, and U.S. Patent Application Publication No. US2016/0076577A1 published Mar. 17, 2016, disclose various pre-molded sealant parts, including seal caps and seal cap assemblies that may be inspected by the method and system of the present invention. While seal caps are shown and described herein, it is to be understood that the method and imaging system of the present invention may be used for the inspection and evaluation of any desired pre-molded sealant part. For example, pre-molded sealant parts, including gaskets, O-rings, shims, washers, grommets, spacers, packing, cushions, mating material, flanges, plugs, and the like, may be inspected and evaluated with the method and imaging system of the present invention.

The pre-molded sealant parts can be formed by any means known in the art, for example, by using injection-filled molds, stamping, male and female molds, extrusion, additive manufacturing, three-dimensional printing or the like, and may be carried out at atmospheric, sub-atmospheric, or super-atmospheric pressures. One skilled in the art knows various methods of forming outer shells 12 of seal caps 10 into a variety of shapes and sizes to fit a particular application. Examples of methods of forming outer shells of seal caps are disclosed in U.S. Pat. No. 7,438,974, the disclosure of which at column 3, line 10 to column 6, line 23 is incorporated herein by reference. Examples of methods of making pre-molded sealant parts using three-dimensional printing are disclosed in PCT/US2020/017417 filed Feb. 10, 2020, the disclosure of which at paragraph [213] to paragraph [243] is incorporated herein by reference. In accordance with the present invention, the pre-molded sealant parts may be any suitable size. For example, the outer shell 12 of the seal cap 10 may be any suitable size, for example, the outer shell may have a diameter of from 0.5 to 5 cm, and a height of from 0.5 to 5 or 10 cm.

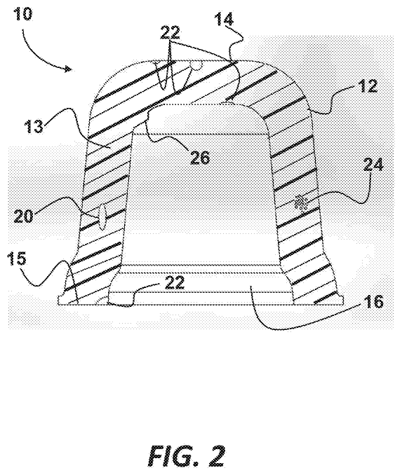

In accordance with the present invention, the outer shell 12 of the seal cap 10 may be comprised of a cured or partially cured adhesive or sealant material. Forming and curing the outer shell 12 of the seal cap 10 may create defects in the form of air bubbles, air cavities, flashing, flaps, open voids, closed voids, lumps, inclusions, porosity, foreign object debris, deformation and the like. Defects in the outer shell of the seal cap may increase the likelihood that the seal cap will fail or may prevent the required fit of the seal cap on a fastener. As schematically shown in FIG. 2, the outer shell 12 of the seal cap 10 may have defects in the form of interior pockets or voids 20, surface pockets or voids 22, air bubbles 24 and/or a deformation defect 26.

Defects in the outer shell 12 of a seal cap 10 may reduce the mechanical properties of the seal cap 10, which may lead to premature failure of the seal cap 10. Interior pockets or voids may expand at altitude and could lead to a rupture of the seal cap 10 resulting in fuel leaks or pressure leaks, may lead to reduced efficacy of the seal cap 10 from preventing electricity from making its way to the metal fastener in the event of a lightning strike, and/or may lead to an increased risk of corrosion. Surface pockets or voids may expand at altitude and could lead to a rupture of the seal cap 10 resulting in fuel leaks or pressure leaks, may lead to reduced efficacy of the seal cap 10 from preventing electricity from making its way to the metal fastener in the event of a lightning strike, and/or may lead to an increased risk of corrosion. Unwanted particles or other foreign object debris may lead to may lead to reduced efficacy of the seal cap 10 from preventing electricity from making its way to the metal fastener in the event of a lightning strike.

In accordance with the present invention, the seal cap 10 can be provided having a cured or partially cured outer shell 12 that may be inspected for defects and may be filled with an uncured sealant portion. The seal cap 10 may typically be inspected by the method and system of the present invention prior to being filled with uncured sealant. However, in some cases, inspection may be performed after the seal cap 10 is filled with sealant.

In accordance with the present invention, the inspection process of the present invention may be performed at ambient temperature conditions after the outer shell 12 of the seal cap 10 is partially or fully cured. The outer shell 12 of the seal cap 10 may be filled after being inspected, or prior to being inspected, with a second quantity of sealant including hydrophobic polymers, and the like. The outer shell and the second quantity of sealant may comprise the same composition. The uncured sealant may be thermally regulated to keep it from becoming cured prior to installation over a fastener. For example, uncured seal caps can be kept at temperatures between and including −100° C. and −25° C. to retard curing, for example, the sealant can be kept at a minimum of −75° C., and for example, at a maximum of −45° C. In accordance with the present invention, the inspection process may be conducted at such reduced temperatures.

The sealant may comprise elastomeric polymers. For example, the sealant may be a one-component or a two-component formulation. For example, the sealant may be comprised of a one-component silicone composition, or butadiene rubber or other synthetic rubbers, such as styrene-butadiene, silicone rubber, siloxane, and acrylonitrile-butadiene and the like, butyl acrylate, and/or 2-ethylhexyl acrylate. The sealant may comprise at least two reactants capable of reacting to form a cured composition. For example, a curable composition can comprise an isocyanate-terminated chain-extended polythioether prepolymer and a polyamine capable of reacting to form a cured polymer. A curable composition may include a catalyst for the curing reaction and other components such as, for example, fillers, pigments, and adhesion promoters. A curable composition may be curable at room temperature or may require exposure to elevated temperature such as a temperature above room temperature or other condition(s) to initiate and/or to accelerate the curing reaction. A curable composition may initially be provided as a two-part composition including, for example, a separate base component and an accelerator component. The base composition can contain one of the reactants participating in the curing reaction such as an isocyanate-terminated chain-extended polythioether prepolymer and the accelerator component can contain the other reactant such as a polyamine. The two components can be mixed shortly before use to provide a curable composition. Alternatively, the polythioether base may contain the polyamine, and the second component may be an epoxy-containing compound and/or an epoxy-adduct.

The sealant composition may be a polythiol, a polyalkenyl, a metal complex, and an organic peroxide. The compositions may comprise a thiol-terminated sulfur-containing prepolymer, a polyalkenyl, a metal complex, and an organic peroxide. For example, the sealant may comprise a thiol-terminated sulfur-containing prepolymer such as a thiol-terminated polythioether prepolymer, a thiol-terminated polysulfide prepolymer, a thiol-terminated sulfur-containing polyformal prepolymer, a thiol-terminated monosulfide prepolymer, or a combination of any of the foregoing. The sealant composition may comprise a thiol/ene curing chemistry. For example, a sulfur-containing prepolymer such as a thiol-terminated polythioether reacting with a divinyl ether. In accordance with the present invention, the sealant composition may be a polysulfide cured with manganese dioxide or magnesium chromate. An amine catalyst can be used, or the reaction may take place via a UV-initiated free-radical reaction.

The sealants may be filled with silica and calcium carbonate to enhance the physical properties of the cured sealants. For example, filler particles or microcapsule may be added to the sealant formulations, for example, to adjust the viscosity of the sealant formulations, to establish the physical properties of a cured pre-molded sealant part, to establish the density of a cured pre-molded sealant part, and/or to establish the electrical and/or thermal properties of a cured pre-molded sealant part. Examples of suitable low-density filler particles or microcapsules include glass particles or microcapsules, polymeric particles or microcapsules, thermally-expanded thermoplastic microcapsules, thermally-expanded microcapsules comprising an exterior coating of an aminoplast resin such as a melamine or a urea/formaldehyde resin, and the like. A low-density filler particle or microcapsule can have a specific gravity, for example, less than 0.5, less than 0.3, or less than 0.1. A sealant material can comprise low density microcapsules.

A low-density filler particle or microcapsule can comprise a thermally expandable microcapsule.

A thermally expandable microcapsule refers to a hollow shell comprising a volatile material that expands at a predetermined temperature. Thermally expandable thermoplastic microcapsules can have an average initial particle size of 5 μm to 70 μm, in some cases 10 μm to 24 μm, or from 10 μm to 17 μm. The term “average initial particle size” refers to the average particle size (numerical weighted average of the particle size distribution) of the microcapsules prior to any expansion. The particle size distribution can be determined using a Fischer Sub-Sieve Sizer or by optical inspection.

Examples of suitable thermoplastic microcapsules include Expancel™ microcapsules such as Expancel™ DE microspheres available from AkzoNobel. Examples of suitable Expancel™ DE microspheres include Expancel™ 920 DE 40 and Expancel™ 920 DE 80. Suitable low-density microcapsules are also available from Kureha Corporation.

Low density filler such as low density microcapsules can be characterized by a specific gravity within a range from 0.01 to 0.09, from 0.04 to 0.09, within a range from 0.04 to 0.08, within a range from 0.01 to 0.07, within a range from 0.02 to 0.06, within a range from 0.03 to 0.05, within a range from 0.05 to 0.09, from 0.06 to 0.09, or within a range from 0.07 to 0.09, wherein the specific gravity is determined according to ISO 787-11. Low density filler such as low-density microcapsules can be characterized by a specific gravity less than 0.1, less than 0.09, less than 0.08, less than 0.07, less than 0.06, less than 0.05, less than 0.04, less than 0.03, or less than 0.02, wherein the specific gravity is determined according to ISO 787-11.

Low density filler such as low-density microcapsules can be characterized by a mean particle diameter from 1 μm to 100 μm and can have a substantially spherical shape. Low-density filler such as low-density microcapsules can be characterized, for example, by a mean particle diameter from 10 μm to 100 μm, from 10 μm to 60 μm, from 10 μm to 40 μm, or from 10 μm to 30 μm, as determined according to ASTM D6913.

Examples of one-part composition materials and a two-part composition materials that may be used for making pre-molded sealant parts using three-dimensional printing are identified in PCT/US2020/017417 filed Feb. 10, 2020, the disclosure of which at paragraph [52] to paragraph [212] is incorporated herein by reference.

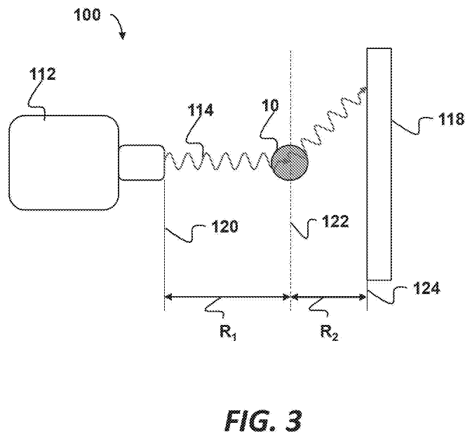

In accordance with the present invention, the pre-molded sealant part inspection process may be performed by an imaging system. FIG. 3 schematically illustrates an imaging system 100 for high-resolution X-ray detection for phase contrast imaging. The imaging system 100 may enable propagation-based X-ray phase contrast imaging (PB-XPC) in a compact, fast manner by approaching PB-XPC from a source and detector perspective. The imaging system 100 may include an X-ray source 112 that directs X-rays (such as in the form of a polychromatic beam 114) towards a pre-molded sealant part 10 that is being imaged. The imaging system 100 further includes a detector 118, located on a side opposite the X-ray source with respect to the pre-molded sealant part 10, to receive, or detect, the X-rays that pass through and are refracted by the pre-molded sealant part 10 through free-space propagation. As shown in FIG. 3, the phase contrast imaging X-ray detector 118 may be planar and may be vertically oriented next to the pre-molded sealant part 10. The X-ray source 112 may be a standard laboratory micro-focus source and the X-ray detector 118 may be a very high resolution and dose efficient X-ray detector having a pixel pitch of less than or equal to 25 microns.

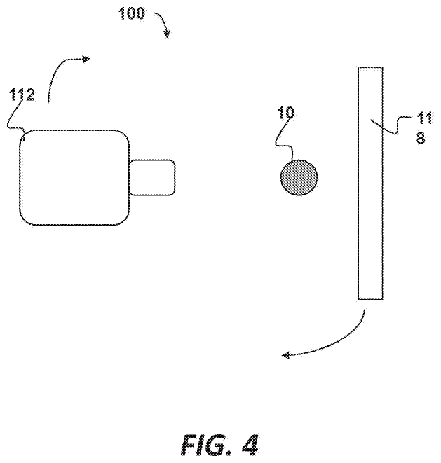

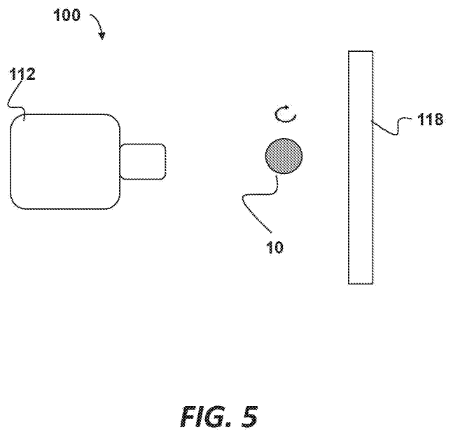

As shown in FIG. 4, the X-ray source 112 and the phase contrast imaging X-ray detector 118 may be rotated about a stationary pre-molded sealant part 10 to allow the imaging system 100 to perform the imaging of the pre-molded sealant part 10. As shown in FIG. 5, the pre-molded sealant part 10 may be rotated between a stationary X-ray source 112 and a stationary phase contrast imaging X-ray detector 118 to allow the imaging system 100 to perform imaging of the pre-molded sealant part 10.

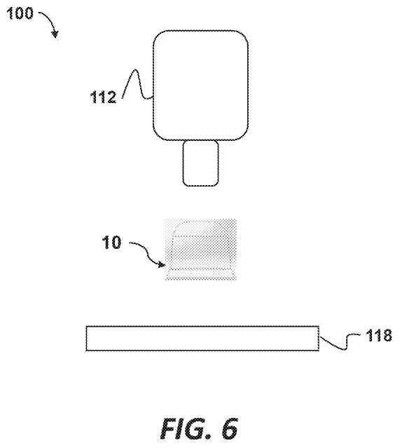

As shown in FIG. 6, the imaging system 100 may include an X-ray source 112 that is provided above or below the pre-molded sealant part 10 to direct X-rays (such as in the form of a polychromatic beam) towards a pre-molded sealant part 10 that is being imaged. As shown in FIG. 6, the phase contrast imaging X-ray detector 118 may be planar and may be horizontally oriented below or above the pre-molded sealant part 10.

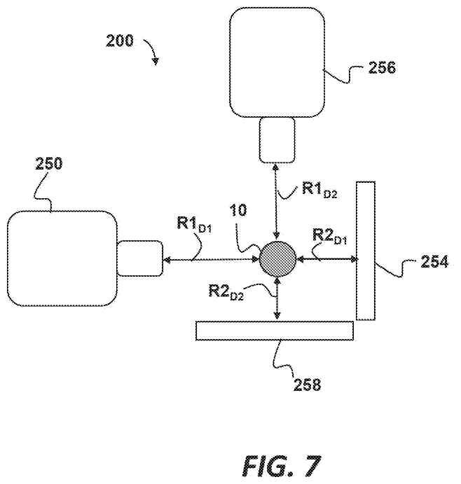

FIG. 7 illustrates a schematic diagram of an imaging system 200 to obtain both multi-spectral and phase retrieval data for PB-XPC. As schematically shown in FIG. 7, the imaging system 200 may include two different X-ray sources in conjunction with two fine-pitch single layer X-ray detectors that are operating in different planes.

As most clearly shown in FIG. 3, an output plane 120 of the focal spot of the X-ray source 112 is located a distance R₁ from the object plane 122 while an image plane 124 of the X-ray detector 118 is a distance R₂ from the object plane 122. A corresponding pixel pitch (for example less than or equal to 25 microns), R₁ (which can be seen as an X-ray source focal spot to object plane/source to object distance) and R₂ (which may be seen as an object plane to detector image plane/object to detector distance) may be selected to achieve, fast, dose efficient PB-XPC using a benchtop device. In accordance with the present invention, the selection of the pixel pitch may be based on the X-ray refraction angle of the X-ray 114 leaving the pre-molded sealant part 10 (calculated from the complex refractive index) and the propagation distance R₂. A small R₂ may be more desirable, leading to a deviation of the X-ray 114 that is detectable by a detector having pixels with a small pixel pitch (such as less than or equal to 25 microns).

In accordance with the present invention, the distance R₁ from the object plane 122 may be less than 10 cm. For example, the distance R₁ from the object plane 122 may typically range from 1 to 50 cm, or from 1 to 25 cm, or from 1 to 10 cm. The distance R₂ may typically range from 0 to 200 cm. The distance R₂ from the object plane 122 may be less than the distance R₁ from the object plane 122.

In accordance with the present invention, the imaging system 100 may detect the minute (in the range of 10⁻⁵-10⁻⁴ rad) X-ray refraction associated with phase changes encoded by the pre-molded sealant part 10.

Imaging systems that may be adapted for use in accordance with the present invention may include digital imaging systems disclosed in U.S. Patent Application Publication No. US2019/0113466 published Apr. 19, 2019, which is incorporated herein by reference.

In accordance with the present invention, the X-ray source 112 may be a standard low-power (8 W) laboratory micro-focus source with a focal spot size of 1 to 30 μm, or from 2.5 to 15 μm, or from 4.5 to 10 μm. The focal spot size may be the size of the X-ray source electron beam that contacts the anode target materials e.g., tungsten or molybdenum, which then produces X-rays that propagate to the pre-molded sealant part 10 and subsequently to the detector 118). When the focal spot is small, the penumbral blur from the extent of the focal spot is minimized or reduced such that that the X-ray source 112 does not limit spatial resolution within the imaging system 100. A coherent or partially coherent incident beam may be used to detect phase changes due to the pre-molded sealant part 10. The lateral coherence length is proportional to the source-to-object distance, R₁, and inversely proportional to the focal spot size. That is, a smaller focal spot may result in a partially coherent beam with a smaller R₁ distance, or in other words, a more compact system.

In accordance with the present invention, the X-ray source 112 generates X-ray radiation, in the form of a set of X-ray beams, that is transmitted toward an object of interest. The X-ray source 112 may generate a polychromatic X-ray beam. The polychromatic X-ray beam may be a micro-focused X-ray beam. For example, the X-ray radiation may have wavelengths of from 0.01 to 10 nanometers. The X-ray source 112 may be stationary or moveable. Any suitable number of X-ray sources 112 may be used to generate the X-ray radiation, e.g., one, two, three or more X-ray sources 112. The X-ray source 112 may generate a relatively low amount of X-ray dosage. The X-ray source 112 may generate X-ray radiation having a single wavelength or multiple wavelengths.

In accordance with the present invention, the X-ray detector 118 may be a high-resolution x-ray detector based using a direct conversion photoconductor and complementary metal-oxide semiconductor (CMOS) pixel electronics having a pixel pitch of less than or equal to 25 microns. The X-ray detector 118 may include a bottom CMOS layer with a plurality of small sized pixels. In accordance with the present invention, the pixel pitch of each of the pixels may be less than or equal to 25 microns. The X-ray detector 118 may also include a stability/blocking layer, a photoconductor layer, a blocking layer and an electrode layer. The X-ray detector 118 may also include a set of bond pads that may be used to enable an electrical connection for control/data signals.

The photoconductor layer of the X-ray detector 118 may be an amorphous selenium (a-Se) photoconductor layer. The blocking layers on either side of the a-Se photoconductor layer may be used to improve mechanical stability of the X-ray detector 118 and/or to reduce the dark current during operation of the X-ray detector 118 at high electric fields. Alternatively, the X-ray detector 118 may include only one or none of the blocking layers.

The stability/blocking layer may be a polyimide layer that may function as both, an anticrystallization layer and as a blocking contact on the bottom of the photoconductor layer. Alternatively, the blocking layer may be a parylene layer that functions as a blocking contact for the photoconductor layer. A contact layer between the photoconductor layer and the stability/blocking layer may also be, but is not limited to, a p-type layer (such as As-doped selenium) or other suitable soft polymer materials. A contact layer between the photoconductor layer and the blocking layer may also be, but is not limited to, a n-type layer such as alkali-metal-doped selenium or cold deposited selenium, or any other suitable organic and inorganic hole blocking layers. Although the previous discussion relates to a direct conversion X-ray detector, other suitable high-resolution detector technologies, such as indirect conversion detectors, or a combination of direct conversion and indirect conversion X-ray detectors may be used.

In direct conversion X-ray detectors, amorphous selenium, silicon, CdZnTe, CdTe, HgI₂, PbO, and scintillator infused organic photoconductors such as perovskite integrated with CMOS or thin-film-transistor (TFT) pixel arrays may be used for the photoconductor layer. With indirect conversion X-ray detectors, CsI, LaBr₃, and pixelated GOS or CsI scintillators integrated CMOS or TFT pixel arrays may be used.

In accordance with the present invention, a very fine, or small, pixel pitch, high dose efficiency direct conversion X-ray detector may be used to work in conjunction with the micro-focus source for the PB-XPC approach.

The phase contrast imaging X-ray detector 118 of the imaging system 100 of the present invention allows the imaging to include added detail of the pre-molded sealant part due to phase contrast. The imaging may allow images to be taken in a few seconds. As such, the imaging system 100 of the present invention may be seen as a highly compact, fast, low dose PB-XPC system. Imaging time can be further reduced by using high output micro-focus X-ray tubes (e.g., metal jet X-ray) as the X-ray source, however, use of a high dose efficiency detector map help further reduce imaging time (e.g., for high throughput industrial applications) and more importantly, to minimize or reduce further radiation damage the pre-molded sealant part being imaged.

In accordance with the present invention, the phase contrast imaging X-ray detector 118 of the imaging system 100 may have a 200 microns or less pixel pitch, for example, a pixel pitch of less than 100 microns, or less than 50 microns, or less than 25 microns, or less than 10 microns.

In accordance with the present invention, multiple phase contrast imaging X-ray detectors 118 may be used to form an array of phase contrast imaging X-ray detectors 118 to allow for the imaging of larger portions of pre-molded sealant part.

The X-ray detector 118 may be planar and may be oriented horizontally, vertically or in any other desired orientation. The X-ray detector 118 may be stationary or moveable. Any suitable number of X-ray detectors 118 may be used to receive the X-ray radiation, e.g., one, two, three or more X-ray detector elements. The object of interest may be held stationary in front of the X-ray detector 118 or may be moved and/or rotated in front of the X-ray 118. The X-ray detector 118 may provide absorption contrast and phase contrast information. The X-ray detector 118 may be capable of increased contrast at relatively low amounts of X-ray radiation. The X-ray detector 118 may have a relatively high spatial resolution.

While a compact phase contrast X-ray detector with direct conversion selenium-CMOS detectors was previously described herein, any other suitable direct conversion materials such as HgI₂, CZT, TIBr, and silicon can be employed in place of selenium and the CMOS pixels could be replaced by poly-Si, metal-oxide, or common II-VI or III-V semiconductors. Moreover, high-resolution indirect-conversion X-ray detectors (e.g., with thin scintillators, or pixelated scintillators) can also be employed albeit likely with lower dose efficiency than direct conversion detectors. Micro-computed-tomography (microCT) is also possible with this system by adding a rotational stage (or creating a rotating gantry) for generating multiple x-ray projection images of the object from different perspectives, and CT reconstruction software.

As shown in FIG. 7, the imaging system 200 may include a first X-ray source 250 that directs a polychromatic beam towards a pre-molded sealant part 10 that is then detected by a first X-ray detector 254. The system may further include a second X-ray source 256 that directs a polychromatic beam towards the pre-molded sealant part 10 that is then detected by a second X-ray detector 258. In accordance with the present invention, the distance between the first X-ray source 250 and the object plane R1D₁ and the distance between the second X-ray source 256 and the object plane R1D₂ may be set to the same value while the distance between the object plane and the image plane of the first X-ray detector 254 R2D₁ and the distance between the image plane of the second X-ray detector 258 and the object plane R1D₂ may be set to different values. The two sets of X-ray source and X-ray detector pairs allow the system to obtain multiple two-dimensional (2D) images from the first and second X-ray detectors. Alternatively, the beams of the first X-ray source and the second X-ray source may be directed towards the pre-molded sealant part 10 in non-parallel directions. The beams of the first X-ray source and the second X-ray source may also be directed towards the pre-molded sealant part 10 in a perpendicular direction.

In accordance with the present invention, where multiple images are generated or detected, they may then be combined in any known methodologies to obtain a single overall image (if required) using reconstruction algorithms.

The imaging system 200 may allow the X-ray spectrum from the first X-ray source 250 and the X-ray spectrum from the second X-ray source 256 to be defined independently of the first X-ray detector 254 and the second X-ray detector 258 leading to additional simplicity in the reconstruction algorithms. In accordance with the present invention, the imaging system of FIG. 7 may enable acquisition of phase contrast images, phase retrieval, multi-spectral images and conventional attenuation images in a single scan. To obtain a three-dimensional (3D) image, either the object or the source/detector pairs can be rotated to obtain multiple projections for reconstruction or further X-ray source/X-ray detector pairs may be used.

The imaging systems 100 and 200 may be operated in accordance with technical specifications published by the manufacturer. For example, the X-ray source and the X-ray detector may be operated using selected parameters known to those skilled in the art such as X-ray dosage, integration time, energy spectrum range, wavelength(s), frame rate, scanning speed, pixel size, pixel pitch, X-ray phase contrast, rotational stage stepping, power supply and the like.

The imaging systems of the present invention may provide fast imaging in a compact system and allows micro-anatomical imaging to visualize a greater level of detail and avoid damaging by using less X-ray radiation to acquire an image. The combination of better visualization of pre-molded sealant parts using phase contrast X-ray and high detector dose efficiency may allow high resolution, non-invasive and non-destructive imaging for the pre-molded sealant parts.

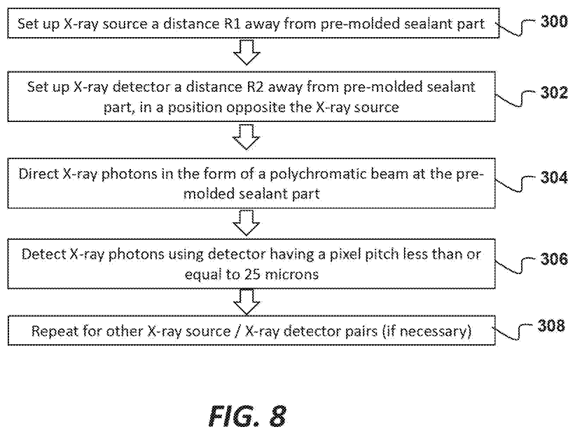

FIG. 8 is a flow chart illustrating a method of phase contrast imaging a pre-molded sealant part. Initially, an X-ray source is placed a distance R₁ away from the pre-molded sealant part being imaged (300). This distance may be less than 10 cm and, may be measured from the focal spot of the X-ray source to the object plane of the pre-molded sealant part. An X-ray detector is then placed a distance R₂ from the object (302) on a side of the pre-molded sealant part opposite the location of the X-ray source. This distance may be between 0 cm and 200 cm and may be measured from the object plane to a detector plane. The X-ray source then directs a polychromatic beam towards the pre-molded sealant part (304). The resulting photons are then detected by the X-ray detector via its set of pixels that are sized to be less than or equal to 25 microns (306). If necessary, further X-ray source and X-ray detector pairs may be placed (308) around the pre-molded sealant part to obtain multiple images with a lower radiation dose.

In accordance with the present invention, a method of inspecting a pre-molded seal cap comprises positioning the seal cap between an X-ray source and an X-ray detector. In accordance with the present invention, the seal cap may be positioned manually or automatically between the X-ray source and the X-ray detector. For example, the seal cap may be brought into the proximity of the X-ray detector by placing the seal cap on or near the detector, such as by supporting the seal cap on a horizontally orientated planar detector array, or the seal cap may be brought near the detector by a robotic arm, a conveyor belt or the like. The X-ray source may be positioned vertically above the X-ray detector and seal cap to be inspected. Alternatively, the seal cap shell may be positioned adjacent or next to a vertically orientated planar X-ray detector array and X-ray source.

In accordance with the present invention, the image that is produced by the imaging system can then be viewed on a display of a computer or computing system. The resolution of the imaging system may be selected as desired. For example, when inspecting for defects within the pre-molded sealant part, the image that is produced by the imaging system may be reviewed for defects having a size less than or equal to about 1 millimeter, or about 100 micrometers, or about 50 micrometers, or about 25 micrometers, or about 10 micrometers, or about 1 micrometer, or about 500 nanometers, or about 100 nanometers. In addition to visual displays of the images, the image data from the imaging system may be analyzed by commercially available computer software programs known to those skilled in the art. The image data may allow for defects to be identified in pre-molded sealant parts comprising a high-volume percentage low-density sealant composition including thermally expanded thermoplastic microcapsules. The image data provided by the imaging system may allow defects to be correctly identified and distinguished from the microcapsules and the like.

In accordance with the present invention, a pre-molded sealant part may be inspected rapidly, for example, in less than 10 seconds, or less than 5 seconds, or less than 2 seconds, or less than 1 second.

In accordance with the present invention, the defects identified by the imaging system are analyzed to determine if they are acceptable or cause the pre-molded sealant part to be rejected. The size and/or the location of each identified defect may cause a pre-molded sealant part to be accepted or rejected. For example, an identified defect having a size of less than 750 microns, or less than 500 microns, or less than 300 microns, may be considered acceptable. An identified defect having a size of greater than 300 microns, or greater than 500 microns, or greater than 750 microns may be considered defective. The location of the identified defect may also be evaluated in determining if the pre-molded sealant part is to be accepted or rejected.

The following Examples are intended to illustrate various aspects of the present invention and are not intended to limit the scope of the invention.

### Example 1

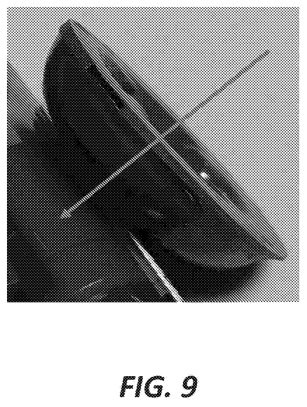

A seal cap as shown in FIG. 9 was inspected for defects using an imaging system in accordance with the present invention. The seal cap was formed using a standard molding process with a sealant material comprising polysulfide sealant. The seal cap had a concave outer shell forming an internal cavity. The outer shell of the seal cap has an outer diameter of 22.8 mm and a height of 6.5 mm. The internal cavity of the outer shell of the seal cap was not filled with a second sealant material as shown in FIG. 9. As shown in FIG. 9, the outer shell of the seal cap comprises a radially outermost outer lip at the bottom face of the outer shell. An open surface void defect was created in the seal cap having an approximate height of 0.5 mm, an approximate length of 3.8 mm, and an approximate depth of 1.3 mm. The seal cap was inspected using an imaging system to detect the surface void defect and to identify any closed voids or additional internal defects. The imaging system used to perform the inspection was a publicly disclosed prototype from KA Imaging Inc. under the designation Libra. The imaging system is operated in accordance with technical specifications published by the manufacturer.

The imaging system comprises a standard low-power (8 W) laboratory micro-focus source as the X-ray source. The X-ray source is a tungsten target PXSS-927-LV micro-focus source (Thermo Fisher Scientific) having a variable beam quality of 20 to 60 kV tube potential, a maximum 0.180 mA tube current, and a maximum 8 W power output. The focal spot size varies approximately linearly with power from 5 to 9 μm. There is no inherent filtration by the X-ray source with the exception of the 254 μm Beryllium window. The imaging system also comprises a contrast imaging X-ray detector with direct conversion selenium-CMOS detectors. The contrast imaging X-ray detector has a pixel pitch of 7.8 μm.

A micro-focused X-ray beam is directed from the X-ray generating source at the bottom of the outer shell of the seal cap in the X-ray propagation direction identified in FIG. 9. The refracted X-rays that pass through the seal cap are detected by the contrast imaging X-ray detector to produce images of several different areas of the outer shell of the seal cap.

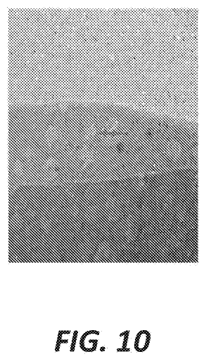

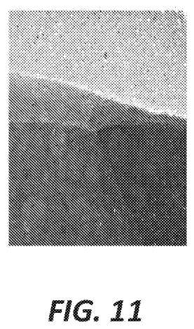

Grayscale images of the images generated by the imaging system are shown in FIGS. 10 and 11.

FIG. 10 shows a ten-frame average of images having about a 0.82 mm width and including a portion of the seal cap taken in the X-ray propagation direction identified in FIG. 9. The imaging system used R₁=18 cm, R₂=8 cm, and a 200 ms integration time obtain the images. As shown in FIG. 10, the lightest top section of the image is air, the adjacent middle section is the sealant of the outer lip of the outer shell of seal cap, and the darkest bottom section of the image is the outer lip and body of the outer shell of the seal cap. FIG. 10 also shows microcapsules that have been added to the sealant of the seal cap to reduce the weight of the sealant. The microcapusle has a size of about 87 μm.

FIG. 11 shows a ten-frame average of images having about a 0.62 mm width and including a portion of the seal cap taken in the X-ray propagation direction identified in FIG. 9. The imaging system used R₁=18 cm, R₂=16 cm, and a 400 ms integration time to obtain the images. As shown in FIG. 11, the lightest top section of the image is air, the adjacent middle section is the sealant of the outer lip of the outer shell of seal cap, the darker bottom left section of the image is the sealant of the outer lip and body of the outer shell of the seal cap, and the darkest bottom right section of the image is the open surface void defect. As shown in FIG. 11, the open surface void defect is located in both the outer lip and the body of the seal cap, and may extend to the outer circumference of the seal cap.

For purposes of the detailed description, it is to be understood that the invention may assume various alternative variations and step sequences, except where expressly specified to the contrary. Moreover, other than in any operating examples, or where otherwise indicated, all numbers such as those expressing values, amounts, percentages, ranges, subranges and fractions may be read as if prefaced by the word “about,” even if the term does not expressly appear. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques. Where a closed or open-ended numerical range is described herein, all numbers, values, amounts, percentages, subranges and fractions within or encompassed by the numerical range are to be considered as being specifically included in and belonging to the original disclosure of this application as if these numbers, values, amounts, percentages, subranges and fractions had been explicitly written out in their entirety.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. In this application, the articles “a,” “an,” and “the” include plural referents unless expressly and unequivocally limited to one referent.

As used herein, “including,” “containing” and like terms are understood in the context of this application to be synonymous with “comprising” and are therefore open-ended and do not exclude the presence of additional undescribed or unrecited elements, materials, ingredients or method steps. As used herein, “consisting of” is understood in the context of this application to exclude the presence of any unspecified element, ingredient or method step. As used herein, “consisting essentially of” is understood in the context of this application to include the specified elements, materials, ingredients or method steps “and those that do not materially affect the basic and novel characteristic(s)” of what is being described.

As used herein, the terms “on,” “onto,” “applied on,” “applied onto,” “formed on,” “deposited on,” “deposited onto,” mean formed, overlaid, deposited, or provided on but not necessarily in contact with the surface. For example, an electrodepositable coating composition “deposited onto” a substrate does not preclude the presence of one or more other intervening coating layers of the same or different composition located between the electrodepositable coating composition and the substrate.

Whereas particular embodiments of this invention have been described above for purposes of illustration, it will be evident to those skilled in the art that numerous variations of the details of the present invention may be made without departing from the invention as defined in the appended claims.

1. A method of inspecting a pre-molded sealant part to identify defects in the pre-molded sealant part, the method comprising:
directing a micro-focused X-ray beam through a pre-molded sealant part comprising a sealant material to produce a phase-shifted refracted X-ray beam; and
detecting the refracted X-ray beam with at least one phase contrast imaging X-ray detector to produce an image of the sealant material.

2. The method of claim 1, wherein the pre-molded sealant part comprises a seal cap, a gasket, an O-ring, a shim, a washer, a grommet, a spacer, a packing, a cushion, a mating material, a flange or a plug.

3. The method of claim 1, wherein the pre-molded sealant part comprises a seal cap.

4. The method of claim 3, wherein the seal cap comprises a cured or partially cured outer shell.

5. The method of claim 1, wherein the pre-molded sealant part is made by molding, extrusion, additive manufacturing or 3D printing.

6. The method of claim 1, wherein the defects identified in the pre-molded sealant part comprise air bubbles, air cavities, flashing, flaps, open voids, closed voids, lumps, inclusions, porosity, deformation or foreign object debris.

7. The method of claim 1, wherein the defects identified in the pre-molded sealant part comprise surface voids and interior voids.

8. The method of claim 1, wherein the defects in the pre-molded sealant part have a size of from 100 nm to 1 mm.

9. The method of claim 1, wherein the defects in the pre-molded sealant part have a size of less than or equal to 1 mm.

10. The method of claim 1, wherein the defects in the pre-molded sealant part have a size of less than or equal to 300 microns.

11. The method of claim 1, wherein the scanning of the pre-molded sealant part is performed at ambient temperature conditions.

12. The method of claim 4, wherein the cured or partially cured outer shell cap is filled with uncured sealant prior to the scanning.

13. The method of claim 1, wherein the at least one phase contrast imaging X-ray detector is vertically oriented, and wherein the pre-molded sealant part is interposed adjacent to the vertically oriented at least one phase contrast imaging X-ray detector.

14. The method of claim 1, wherein the at least one phase contrast imaging X-ray detector is horizontally oriented, and wherein the pre-molded sealant part is interposed on or above the horizontally oriented at least one phase contrast imaging X-ray detector.

15. The method of claim 1, wherein the pre-molded sealant part is moved during the scanning of the pre-molded product.

16. The method of claim 1, wherein the micro-focused X-ray beam is provided by at least one X-ray source.

17. The method of claim 1, wherein the at least one X-ray generating source and the at least one phase contrast imaging X-ray detector are moved around the pre-molded product during the scanning of the pre-molded product.

18. The method of claim 1, further comprising directing a second micro-focused X-ray beam through a pre-molded sealant part comprising a sealant material to produce a second phase-shifted refracted X-ray beam; and
detecting the second refracted X-ray beam with a second one of the phase contrast imaging X-ray detectors.

19. The method of claim 1, wherein any defects contained in the pre-molded sealant part are identified in less than 10 seconds.

20. The method of claim 1, further comprising analyzing the image of the pre-molded sealant part on a display of a computing system to identify defects.

21. The method of claim 1, further comprising analyzing the image of the pre-molded sealant part with a computer software program to identify defects.

22. A system for inspecting a pre-molded sealant part to identify defects in the pre-molded sealant part comprising:
at least one X-ray source structured and arranged to direct a micro-focused X-ray beam through a pre-molded sealant part comprising a sealant material to produce a phase-shifted refracted X-ray beam; and
at least one phase contrast imaging X-ray detector structured and arranged to detect the refracted X-ray beam to produce an image of the sealant material.
